(19) United States
(12) Patent Application Publication
(10) Pub. No.: US 2024/0336886 A1
(43) Pub. Date: Oct. 10, 2024

(54) BAFFLE FOR MICROCAVITY CELL CULTURE VESSELS

(71) Applicant: CORNING INCORPORATED, Corning, NY (US)

(72) Inventors: Thomas Albert Cloutier, Arundel, ME (US); William Joseph Lacey, North Andover, MA (US); Ana Maria del Pilar Pardo, Portsmouth, NH (US); Allison Jean Tanner, Portsmouth, NH (US)

(21) Appl. No.: 18/292,184

(22) PCT Filed: Jul. 29, 2022
(86) PCT No.: PCT/US2022/038751

§ 371 Date: Jan. 25, 2024

Related U.S. Application Data
(60) Provisional application No. 63/227,679, filed on Jul. 30, 2021.

(51) Int. Cl. C12M 1/00; C12M 1/24; C12M 1/32; C12M 3/00

(52) U.S. Cl. CPC C12M 27/20; C12M 23/08; C12M 23/12; C12M 23/34; C12M 23/44

(57) ABSTRACT

A baffle (130) configured to be disposed in a cell culture vessel is provided. The cell culture vessel (100) may comprise a vessel body (101) comprising a bottom wall (110) and a plurality of side walls (107) that define a cell culture chamber, the bottom wall comprising a cell culture surface (111). The baffle (130) may be configured to be disposed parallel to the cell culture surface (111) within the vessel body, the baffle comprising a first baffle crossbar (133) extending across a length of the cell culture surface; a second baffle crossbar intersecting (135) the first baffle crossbar; and a third crossbar disposed (137) at an end of the first baffle crossbar.

### CROSS-REFERENCE TO RELATED APPLICATIONS

This application claims the benefit of priority under 35 U.S.C. § 119 of U.S. Provisional Application Ser. No. 63/227,679 filed on Jul. 30, 2021, the content of which is relied upon and incorporated herein by reference in its entirety.

### TECHNICAL FIELD

The present specification generally relates to cell culture vessels used for growing cells, more specifically, to microcavity cell culture vessels with a baffle to restrict liquid motion in the cell culture vessel.

### BACKGROUND

Generally, three-dimensional (3D) cell cultures can be better suited for simulating an environment of natural tissues and organs than two-dimensional (2D) cell cultures grown in monolayers. Cells grown in 3D cell cultures are able to form spheroids or cell aggregates by attaching to other deposited cells within the three-dimensional environment, thereby creating a more natural interaction between the cells than 2D cells grown in a monolayer. This arrangement of cells provides a flexible configuration, similar to that of natural tissues. Providing an accurate exemplification of a tissue microenvironment is desirable In order to increase accuracy when conducting experimental research for developing therapies against diseases, it is desirable to develop the therapies in a 3D culture instead of 2D culture, as 3D culture more closely resembles the environment in which the developed drug will ultimately be applied in.

However, the formed spheroids or cell aggregates are susceptible to damage, such as when transporting the cell culture vessel or during media exchange. Transporting a 3D cell culture vessel generally causes the liquid contained therein to move unintentionally, resulting in turbulence within the vessel. Turbulence may also occur when liquid media is added or removed from the vessel during the 3D cell culture process. The turbulence may cause the spheroids or cell aggregates to slosh around or be displaced from the respective microcavities in which they are formed within the 3D culture vessel, since the cells in a 3D cell culture are not attached to any surface of the vessel. If spheroids are displaced from their respective microcavities, the formed spheroids may attach to other formed spheroids, resulting in loss of uniformity of the spheroids within the vessel and the size thereof. Accordingly, a need exists for stabilizing liquid motion within 3D spheroid culture vessels.

### SUMMARY

According to an embodiment, a cell culture vessel comprises a vessel body that defines a cell culture chamber enclosed between a bottom wall, a top wall, and a plurality of side walls extending between the top wall and the bottom wall. The cell culture vessel may comprise a port disposed at one side wall. Optionally, the cell culture vessel may comprise a cap or a lid that is removable to access the port. The bottom wall comprises a cell culture surface, wherein the vessel body is configured to receive a cell culture medium from the port such that the medium is deposited along the cell culture surface. The cell culture vessel further comprises a baffle extending across a width and length of the cell culture surface.

According to an embodiment, a cell culture vessel comprises a vessel body that defines a cell culture chamber between a bottom wall and a plurality of side walls extending upwards from the bottom wall. The cell culture vessel may comprise a port disposed at one side wall. Optionally, the cell culture vessel may comprise a cap or a lid that is removable to access the port. The bottom wall comprises a cell culture surface, wherein the vessel body is configured to receive a cell culture medium from the port such that the medium is deposited along the cell culture surface. The cell culture vessel further comprises a baffle extending across a width and length of the cell culture surface.

According to an embodiment, a baffle configured to be disposed in a cell culture vessel is provided. The baffle may comprise a first baffle crossbar, a second baffle crossbar, and a third baffle crossbar. The first baffle crossbar extends parallel to the cell culture surface along a length of the cell culture surface. The second baffle crossbar extends parallel to the cell culture surface along a width of the cell culture surface and intersects and is perpendicular to the first baffle crossbar. The third baffle crossbar extends parallel to the cell culture surface and is disposed at a port end of the first baffle crossbar, perpendicular to the first baffle crossbar extending across at least a part of a width of the cell culture surface.

In some embodiments, the third baffle crossbar comprises feet, such as a foot extending downward to contact the cell culture surface on each end of the third baffle crossbar. Said feet may be the only points of the baffle that contact the cell culture surface, thereby allowing for a gap between the bottom of the baffle and the cell culture surface. In some embodiments, the third baffle crossbar comprises rounded shoulders on either end of the top of the third crossbar. In some embodiments, the third baffle crossbar is configured to be a flow or fluid diverter.

In some embodiments, the baffle comprises a plurality of retention tabs configured to be retained in baffle retention areas of a cell culture vessel. In some embodiments, the baffle retention areas are defined by recesses, gaps, or cutouts provided at a top portion of a plurality of side walls of the cell culture vessel. The second baffle crossbar may comprise a horizontal portion with vertical portions extending upwards towards the top portion of the side walls on either end of the horizontal portion, respectively. A retention tab may be disposed at a top of each vertical portion of the second baffle crossbar, the retention tabs extending horizontally from the vertical portions towards the respective side walls, and the retention tabs configured to fit within or be disposed or retained within corresponding baffle retention areas of the side walls.

In some embodiments, the first baffle crossbar comprises a horizontal portion extending from an end proximal to the port to an end distal to the port, wherein the end distal to the port further comprises a vertical portion extending upwards towards the top portion of the side wall. A retention tab may be disposed at a top of the vertical portion of the first baffle crossbar, the retention tab extending horizontally from the vertical portion towards the side wall, and the retention tab configured to fit within or be disposed or retained within a corresponding baffle retention area of the side wall.

According to an embodiment, a microcavity cell culture system comprises a cell culture vessel and a baffle disposed within a body of the cell culture vessel. In some embodiments, the microcavity cell culture system further comprises a support or carrier for the cell culture vessel. Optionally, the cell culture vessel may comprise a cap or a lid. The cell culture vessel comprises a cell culture surface, and the cell culture surface may comprise a plurality of microcavities. In some embodiments, the cell culture vessel is a microcavity cell culture flask. In some embodiments, the cell culture vessel is a microcavity open well cell culture plate.

In as aspect, a cell culture vessel is provided. The cell culture vessel comprises a vessel body comprising a bottom wall and a plurality of side walls that define a cell culture chamber, the bottom wall comprising a cell culture surface. A baffle is configured to be disposed parallel to the cell culture surface within the vessel body, the baffle comprising a first baffle crossbar extending across a length of the cell culture surface; a second baffle crossbar intersecting the first baffle crossbar; and a third crossbar disposed at an end of the first baffle crossbar.

In an aspect, the second baffle crossbar is disposed perpendicular to the first baffle crossbar. In an aspect, the second baffle crossbar extends across a width of the cell culture surface.

In an aspect, the third crossbar is disposed perpendicular to the first baffle crossbar. In an aspect, the third baffle crossbar extends across a portion of a width of the cell culture surface. In an aspect, the third baffle crossbar is a flow diverter. In an aspect, the third baffle crossbar comprises rounded shoulders on a top of the third baffle crossbar at either end of the third baffle crossbar.

In an aspect, the third baffle crossbar comprises feet in contact with the cell culture surface to provide a gap where the horizontal portion of the third baffle crossbar is raised away from the cell culture surface. In an aspect, the feet of the third baffle crossbar are the only points of contact the baffle has with the cell culture surface.

In an aspect, the cell culture surface comprises a plurality of microcavities.

In an aspect, the cell culture vessel further comprises a port disposed at one side wall of the plurality of side walls, wherein the port is configured for aspiration and media exchange. In an aspect, the third baffle crossbar is arranged proximal to the port and perpendicular to fluid flow from the port. In an aspect, the cell culture vessel further comprises a cap or lid removable to provide access to the port.

In an aspect, the vessel body further comprises a top wall.

In an aspect, the cell culture vessel further comprises a lid configured to releasably attach to a top portion of the sidewalls to enclose the cell culture chamber.

In an embodiment, a plurality of baffle retention areas are disposed at a top portion of side walls of the vessel. In an embodiment, the baffle further comprises a plurality of retention tabs configured for insertion into corresponding baffle retention areas. In an embodiment, the first baffle crossbar comprises a horizontal portion and a vertical portion, wherein a retention tab is disposed at a top of the vertical portion. In an embodiment, the second baffle crossbar comprises a horizontal portion and vertical portions on either end of the horizontal portion, wherein a retention tab is disposed at a top of each vertical portion.

In an embodiment, the cell culture vessel comprises a microcavity cell culture vessel. In an embodiment, the cell culture vessel comprises a microcavity flask. In an embodiment, the cell culture vessel comprises a microcavity open well plate.

In an embodiment, a bottom of the baffle is raised about 0.04 in to about 0.5 from the cell culture surface.

In an embodiment, an end of the first baffle crossbar is angled to correspond to a draft angle of a corresponding side wall of the cell culture vessel.

In an embodiment, the baffle is formed from a polymer material.

In an embodiment, the vessel body is configured to receive a liquid culture medium within the cell culture chamber, wherein the baffle is configured to inhibit movement of the liquid culture medium across the cell culture surface.

In an aspect, a baffle configured to be disposed in a cell culture vessel is provided. In an embodiment, the baffle is removable.

In an aspect, a cell culture system is provided. In an embodiment, the cell culture system is a microcavity cell culture system. The cell culture system comprises a cell culture vessel and baffle according to embodiments described herein; and a protective carrier for the cell culture vessel. In an embodiment, the system further comprises a lid or cap for the cell culture vessel.

Additional features and advantages of the cell culture vessels described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 shows a perspective view of a cell culture vessel with a baffle according to one or more embodiments shown and described herein.

FIG. 2 shows a perspective view of a cell culture vessel with baffle according to one or more embodiments shown and described herein.

FIG. 3 shows a top view of a cell culture vessel with a baffle according to one or more embodiments shown and described herein.

FIG. 4 shows a cross-sectional side view taken along line B-B of the cell culture vessel with baffle of FIG. 3.

FIG. 5 shows a cross-sectional side view taken along line C-C of the cell culture vessel with baffle of FIG. 3.

FIG. 6 shows a perspective view of a baffle for a cell culture vessel according to one or more embodiments shown and described herein.

FIG. 7 shows a top view of a baffle for a cell culture vessel according to one or more embodiments shown and described herein.

FIG. 8 shows a cross-sectional side view of the baffle along line A-A of FIG. 7.

FIG. 9 shows a cross-sectional side view of the baffle along line B-B of FIG. 7.

FIG. 10 shows a cross-sectional side view of the baffle along line C-C of FIG. 7.

FIG. 11 shows a perspective view of an assembled cell culture system according to one or more embodiments shown and described herein.

FIG. 12 shows a perspective view of cell culture system components according to one or more embodiments shown and described herein.

FIG. 13 shows a perspective view of cell culture system components according to one or more embodiments shown and described herein.

FIG. 14 shows a perspective view of cell culture system components according to one or more embodiments shown and described herein.

FIG. 15 shows a cross-sectional side view taken along a length of a cell culture vessel with a baffle according to one or more embodiments shown and described herein.

### DETAILED DESCRIPTION

Reference will now be made in detail to various embodiments of cell culture vessels with various stabilizer devices located therein, examples of which are illustrated in the accompanying drawings. Whenever possible, the same reference numerals will be used throughout the drawings to refer to the same or like parts. Directional terms as used herein—for example up, down, right, left, front, back, top, bottom, distal, and proximal—are made only with reference to the figures as drawn and are not intended to imply absolute orientation.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

Liquid in cell culture vessels with an open expanse of volume has freedom to move uncontrolled throughout the open area. If the cell culture vessel is moved or transported, the movement can cause turbulence in the liquid, leading to “sloshing” and potentially spilling of the liquid. In contrast to cells cultured in two dimensional (2D) culture, which attached to the culture surface of the vessel, cells such as spheroids in 3D culture are not attached to the culture surface and thus are susceptible to sloshing and may dislocate from the microcavities in which they are being cultured. Such turbulence and dislocation of spheroids from microcavities may cause loss of spheroids. Because dislocated spheroids may end up settling in microcavities with other spheroids, the size of the spheroids can become heterogeneous, resulting in the undesirable loss of uniformity of spheroids.

According to embodiments described herein, a baffle is provided that will stabilize the motion of liquid in a cell culture vessel having an open area that would ordinarily permit turbulence to occur as the vessel is moved. The baffle may be adjusted to fit into different types of cell culture vessels, including microcavity flasks and open-well plates. In some embodiments, the baffle comprises feet. The feet allow the baffle to sit or rest in close proximity to the culture surface of the vessel. In some embodiments, the baffle comprises retention tabs to help to maintain the position of the baffle in the vessel by suspending or hanging the baffle from the retention tabs near the top portion of the inner surface of sidewalls of the vessel. In some embodiments, the retention tabs of the baffle may be secured in place within the baffle retention areas on the side walls by a lid or top wall.

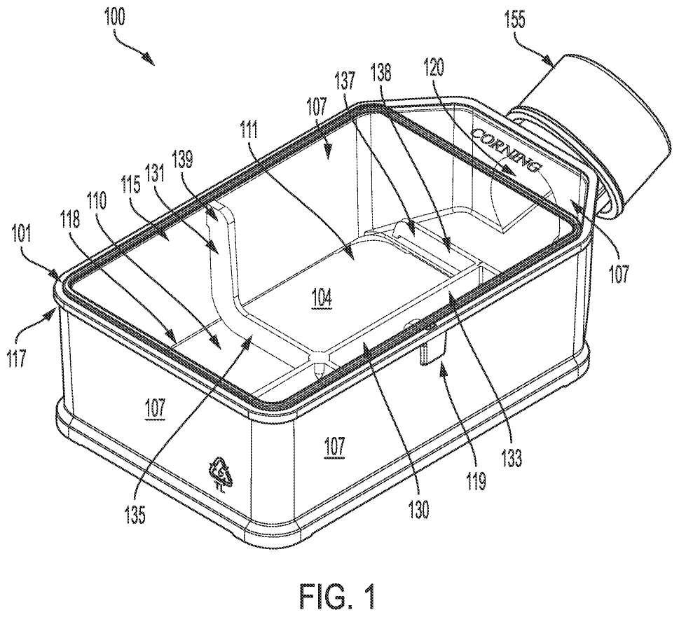

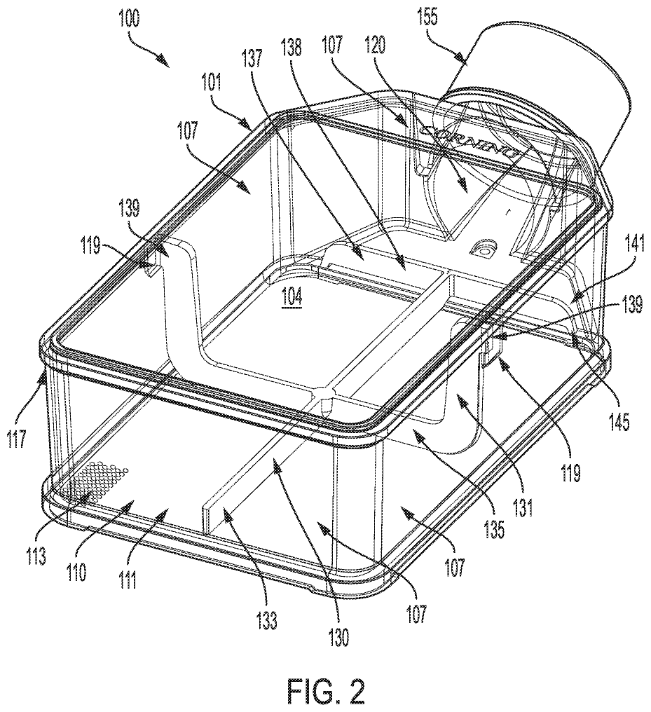

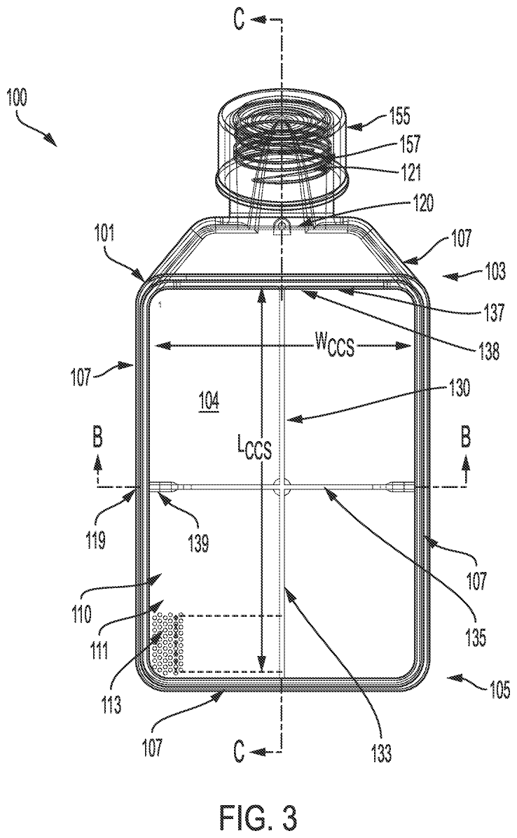

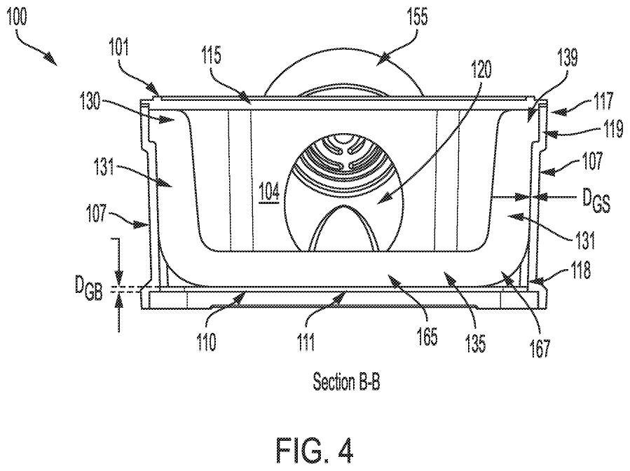

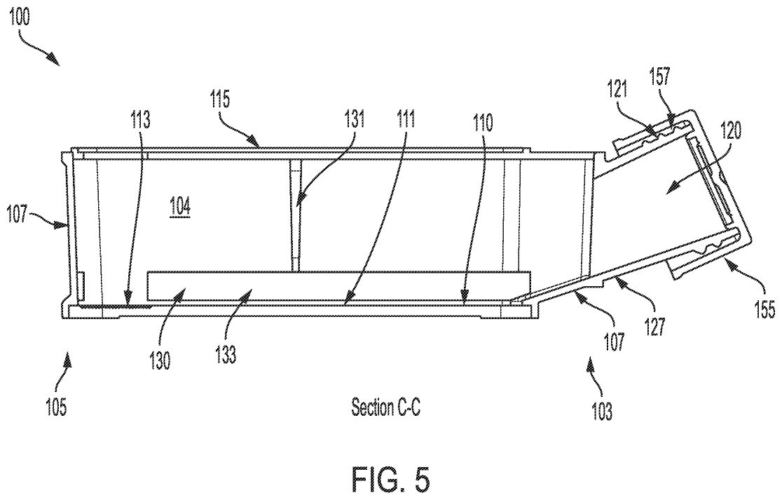

Referring now to FIGS. 1-5, an embodiment of a cell culture vessel 100 with a baffle 130 disposed therein is shown. The cell culture vessel shown in FIGS. 1-5 is a microcavity flask. FIG. 1 shows a perspective view of the cell culture vessel with the baffle disposed therein wherein exterior views of side walls of the vessel are shown and the top wall of the vessel is transparent. FIG. 2 shows a perspective view of a cell culture vessel with the baffle disposed therein wherein the side walls and top wall are shown as transparent to see the placement of the baffle within the vessel. FIG. 3 shows a top view of a cell culture vessel with a baffle disposed therein. FIG. 4 shows a cross-sectional side view taken along line B-B of FIG. 3. FIG. 5 shows a cross-sectional side view taken along line C-C of FIG. 3.

Cell culture vessel 100 has a vessel body 101 that defines a cell culture chamber 104 formed within vessel body 101. In particular, vessel body 101 of cell culture vessel 100 comprises a top wall 115, a bottom wall 110, and a plurality of sidewalls 107 extending between top wall 115 and bottom wall 110. The side walls may have a top portion 117 near the top wall 115 and a bottom portion 118 near the bottom wall 110. The cell culture vessel may have a height H_{CCV}.

Vessel body 101 may further include a port 120 at a side wall 107 of the vessel 100, thereby defining a port end 103 of the vessel 100 proximal to the port 120 and an opposite end 105 of the vessel 100 distal to the port 120. The port 120 is configured to provide fluid access to the cell culture chamber 104. The cell culture vessel 100 is operable to receive a liquid medium into cell culture chamber 104 via port 120. The port 120 may optionally be configured to releasably connect with a cap 155 by any suitable means, such as by threads 157 on the cap 155 configured to releasably interlock with threads 121 on the port 120. In some embodiments, the port may further include a neck 127 or slanted portion directing flow to the bottom wall 110 or cell culture surface 111.

The cell culture vessel 100 may include a cell culture surface 111 positioned along bottom wall 110 of vessel body 101. Cell culture surface 111 may comprise a substrate that includes a plurality of microcavities 113 sized and shaped to receive at least one cell or spheroid therein. Accordingly, cell culture surface 111 is a cell culturing area that is configured to facilitate growth and development of the cells within cell culture chamber 104.

In use, a plurality of cells may be deposited within a cell culture vessel 100 such that cell culture chamber 104 is operable to house the cells within the plurality of microcavities 113 of a cell culture surface 111. With the plurality of cells received along the microcavity substrate 113 of the cell culture surface 111, the development of the cells is facilitated by exposing the cell culture surface 111 to various nutrients and growth fluids during a liquid culture medium filling operation. In particular, a liquid culture medium is deposited within cell culture chamber 104 of the vessel 100 by opening a lid or cap 155 to thereby facilitate access to a port 120 of the cell culture vessel 100. Liquid culture medium may be inserted into the cell culture chamber 104 via an entry port 120, thereby transferring liquid culture medium toward the cell culture surface 111.

The cell culture vessel may comprise a baffle 130 disposed therein. The baffle may serve to stabilize the motion of liquid in the cell culture vessel to prevent or reduce turbulence when the vessel is in use, such as during media exchange, or when the vessel is transported. The baffle 130 may comprise a first baffle crossbar 133, a second baffle crossbar 135, and a third baffle crossbar 137. The baffle 130 may further comprise a plurality of retention tabs 139. As shown in FIG. 1, baffle retention areas 119 on side walls 107 of the vessel 100 protrude to allow the side wall thickness to remain the same while allowing for the gap or recess that defines the baffle retention area 119. As shown in FIG. 2, the baffle retention areas 119 are configured to receive or retain the corresponding retention tabs 139 of the baffle 130.

FIG. 3 shows an overhead view or top view of a cell culture vessel comprising a baffle. When positioned within the cell culture vessel 100, the crossbars 133, 135, 137 of the baffle 130 visually divide the growth area or cell culture surface 111 into quadrants. As shown in FIG. 3, the cell culture surface may have a length L_{CCS} and a width W_{CCS}. The cell culture surface may comprise a plurality of microcavities 113, which may be arranged in an array or repeating pattern and may be round or circular in shape, as shown in the bottom left coiner of FIG. 3.

FIG. 4 shows a cross-sectional side view taken along line B-B of the cell culture vessel 100 and baffle 130 shown in FIG. 3. The cross-sectional side view shows a view looking through the cell culture chamber 104 within body 101 of the cell culture vessel 100 towards port 120 with cap 155. FIG. 4 shows the structure of second baffle crossbar 135, which has a horizontal middle portion 165 that extends across at least a part of the width W_{CCS} of the cell culture surface 111, parallel to the cell culture surface 111. The second baffle crossbar 135 has a width that allows for a gap D_{GS} between a vertical portion 131 of the second baffle crossbar 135 and the sidewalls 107. The second baffle crossbar 135 includes vertical portion 131 on either end of the horizontal portion 165, the vertical portion 131 extending substantially vertically along the side walls 107 to a top portion 117 of the side walls 107. The vertical portion 131 comprises a retention tab 139 at the top portion 117 of the sidewalls 107, near the top wall 115 (or near the lid 250, in some embodiments). Each retention tab 139 is a wing or tab extending horizontally from the vertical portion 131 towards the sidewalls 107 and is configured to be inserted in a baffle retention area 119, which is a gap or recess in the sidewalls 107, also shown by the protrusion at the exterior view of the sidewalls in FIG. 1. The baffle retention area 119 is configured to retain the retention tabs 139 of the baffle 130 to secure the baffle 130 in place within the cell culture chamber 104 of the cell culture vessel 100. The baffle retention area 119 in combination with the retention tabs 130, height of the baffle, and height of the feet of the baffle, allows for a gap D_{GB} between a bottom of the baffle and the cell culture surface 111, wherein only the feet of the baffle are in contact with the cell culture surface 111. In some embodiments, the bottom corners 167 of the second baffle crossbar 135 where the horizontal portion 165 transitions to the vertical portion 131 may be rounded. In some embodiments, the topmost inner corners where the vertical portion 131 transitions to the retention tab 139 may be rounded.

FIG. 5 shows a cross-sectional side view taken along line C-C of the cell culture vessel 100 and baffle 130 shown in FIG. 3. The cross-sectional side view shows a view looking through the cell culture chamber 104 within body 101 of the cell culture vessel 100 to a side wall 107. The port 120 is shown to the right, with the port end 103 of the vessel proximal to the port and an opposite end 105 distal to the port 120. The port 120 is shown with cap 155, the threads 157 of cap interlocked with the corresponding threads 121 of the port. The port 120 further comprises a neck 127 or slanted portion that extends downward towards the bottom wall 110. The first baffle crossbar 133 is shown extending from the port end 103 to the opposite end 105 parallel to the bottom wall 110 or cell culture surface 111, the cell culture surface comprising a plurality of microcavities 113. A vertical portion of the second baffle crossbar extends substantially vertically upwards along side wall 107 to top wall 115.

As described in embodiments herein, a baffle positioned in a cell culture vessel stabilizes the fluidity of liquid culture medium within the cell culture chamber of the vessel body, thereby ensuring liquid culture medium is deposited over cell culture surface without experiencing excessive turbulence or movement when the cell culture vessel is moved. Accordingly, baffles described herein are configured to preserve the condition of the cells received within the microcavities of the cell culture surface by inhibiting movement of liquid culture medium within the cell culture chamber. The baffle prevents the liquid culture medium from the flowing over the baffle crossbars and expanding over the entire open volume area of the cell culture surface within the cell culture chamber, thereby minimizing the ability of liquid culture medium to have adequate space to move therein as cell culture vessel is physically transported.

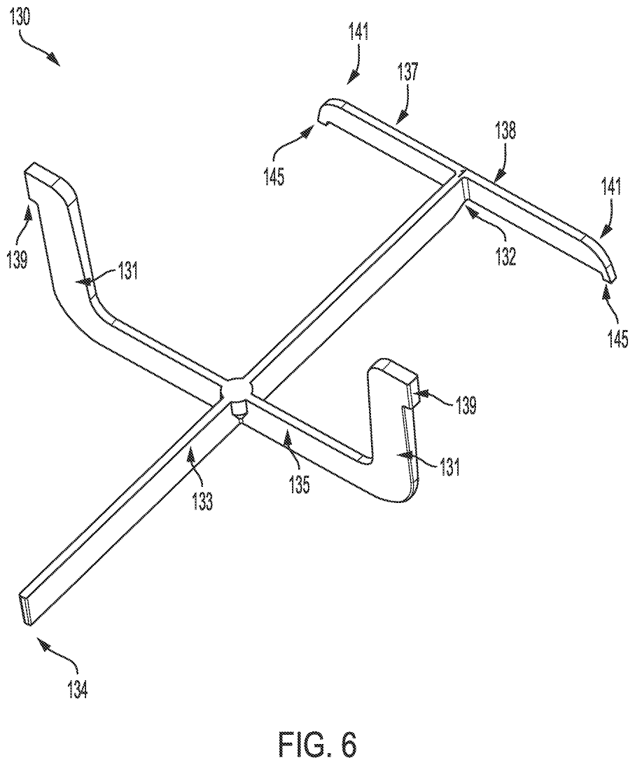

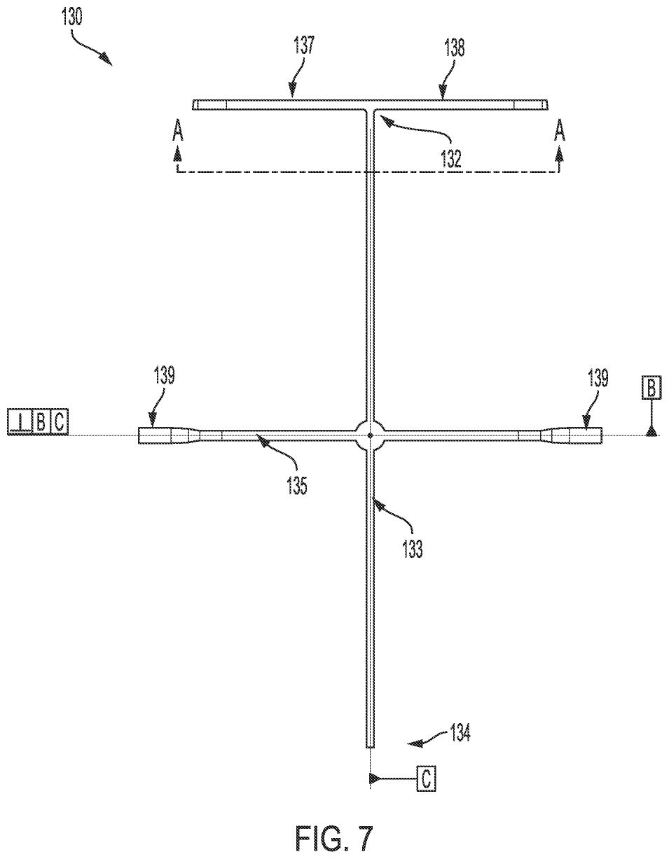

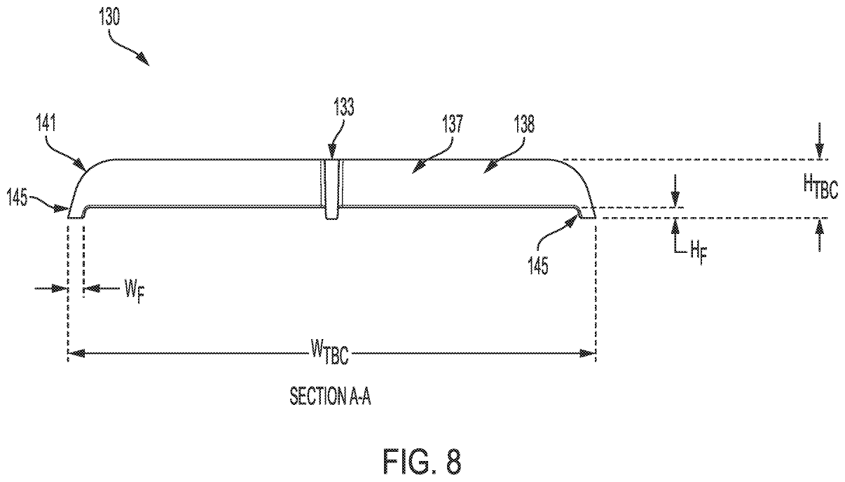

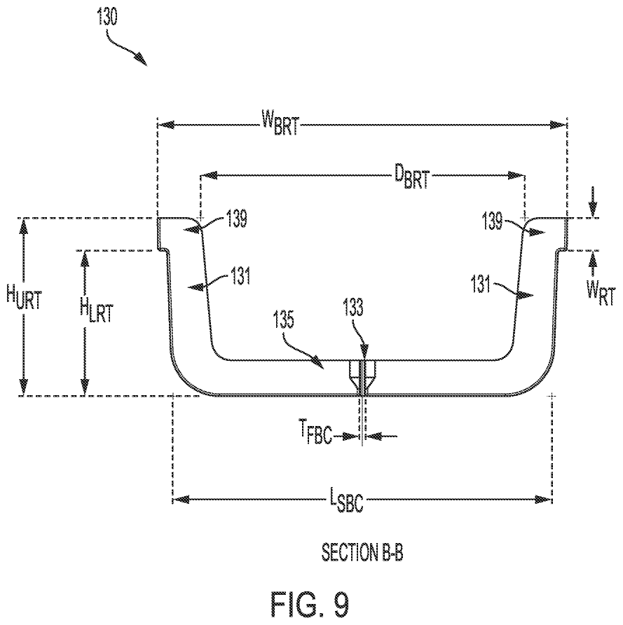

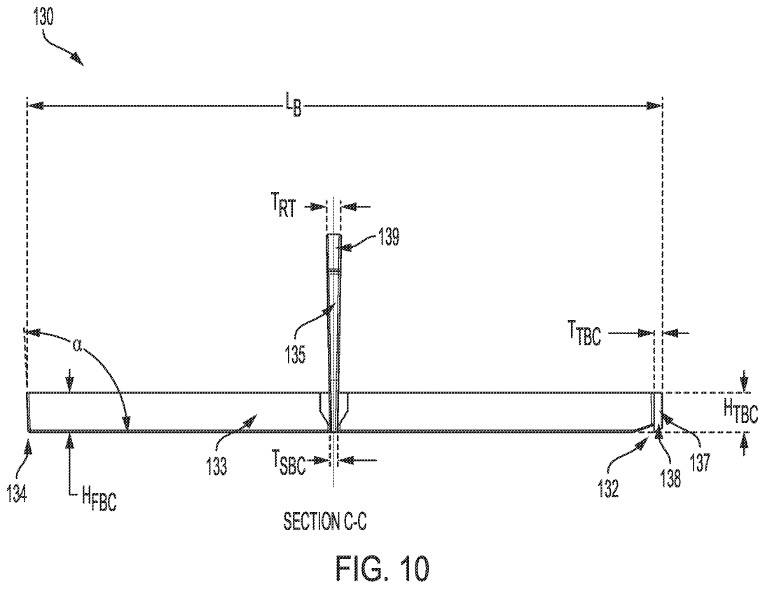

FIGS. 6-10 show an embodiment of a baffle 130 as described herein. FIG. 6 shows a perspective view of a baffle. FIG. 7 shows a top view of a baffle. FIG. 8 shows a cross-sectional side view along line A-A of the baffle of FIG. 7. FIG. 9 shows a cross-sectional side view along line B-B of the baffle of FIG. 7. FIG. 10 shows a cross-sectional side view along line C-C of the baffle of FIG. 7.

In embodiments, the baffle 130 may be sized and shaped to fit within the cell culture chamber 104 such that the baffle crossbars have a width or length that cover a corresponding width or length of cell culture surface 111. The baffle 130 comprises a first baffle crossbar 133, a second baffle crossbar 135, and a third baffle crossbar 137.

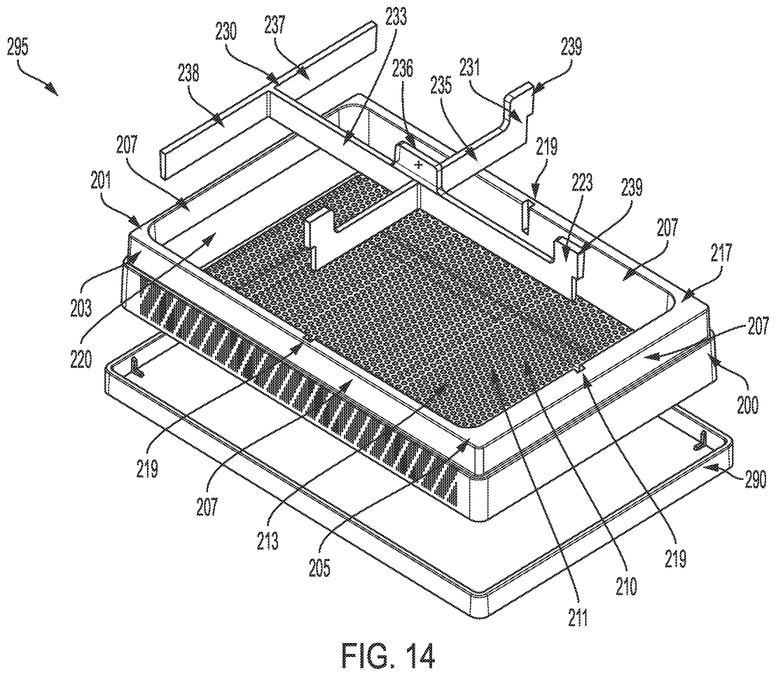

The baffle may have a length L_{B} sized to extend across a length of a cell culture surface within a cell culture vessel. In some embodiments, the L_{B} may correspond to substantially the same as the cell culture surface length L_{CCS}. In some embodiments, the L_{B} is about 4.4 in. The baffle may have a width sized to extend across a width of a cell culture surface W_{CCS} within a cell culture vessel. The baffle may have a height sized to extend from a top of a sidewall towards a cell culture surface to allow for a gap between a bottom of the baffle and the cell culture surface that permits liquid, cells, and/or spheroids to flow underneath the baffle unimpeded. In some embodiments, the baffle 130 may further comprise a handle or pick-up point 236, such as shown in FIG. 14, that allows an area for a user to hold the baffle when inserting the baffle in the vessel or when removing the baffle from the vessel.

The baffle 130 comprises a first baffle crossbar 133 having a port end 132 and an opposite end 134. The first baffle crossbar 133 may be parallel to flow from the port 120 and may extend along a length of the cell culture area 111. The first baffle crossbar may have a height H_{FBC}. In some embodiments, H_{FBC} may be about 0.27 in. The first baffle crossbar may have a thickness T_{FBC}. In some embodiments, the T_{FBC} may be any suitable thickness. In some embodiments, the T_{FBC} is about 0.05 in. In some embodiments, such as shown in FIG. 10, the first baffle crossbar may have an end 134 opposite the port end 132 that comprises an angled edge, wherein α is the angle of opposite end 134 of the first baffle crossbar 133. In some embodiments, α corresponds to a draft angle of the side wall 107 of the cell culture vessel 100 at the end 105 opposite the port end 103. In some embodiments, a is about 91 to about 95 degrees.

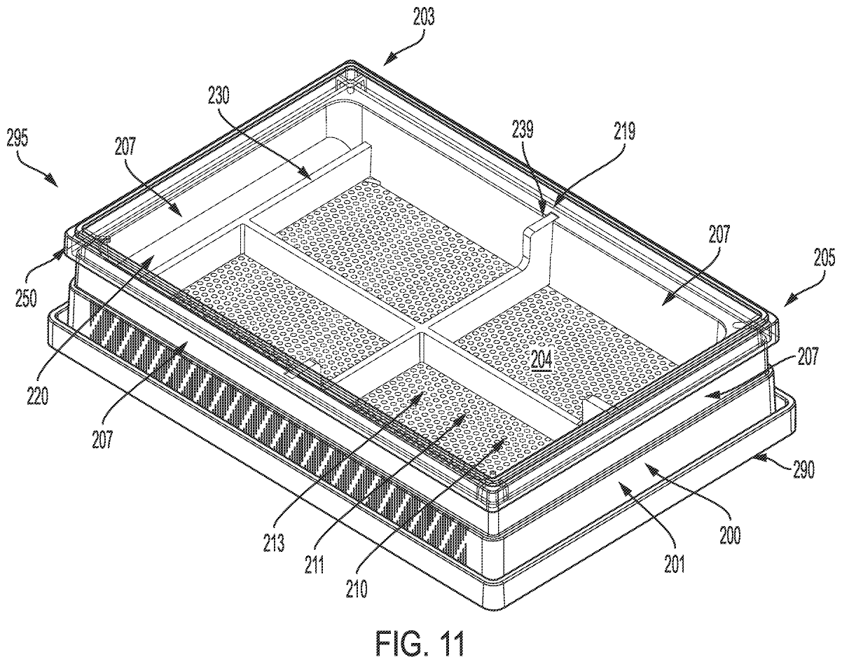

In some embodiments, such as shown in FIGS. 11-15, the first baffle crossbar may have a horizontal portion 225 with a height H_{HPFC} and a vertical portion 223 with a height H_{VPFC}. In some embodiments, the first baffle crossbar 223 may have a vertical portion 223 at an end 205 opposite the port end 203 of the vessel 200, the vertical portion 223 having a retention tab 239 at a topmost portion of the vertical portion 223. The vertical portion 223 may rise substantially vertically up the side wall 207 of the vessel 200 opposite the port end 203, and the retention tab 239 may releasably engage with a recess or baffle retention area 219 at a top 217 of the side wall 207 configured to retain the baffle retention tab 239.

The baffle 130 further comprises a second baffle crossbar 135 intersecting the first baffle crossbar 133 so that the second baffle crossbar 135 is perpendicular to the first baffle crossbar 133. The second baffle crossbar 135 may be horizontal to fluid flow from the port 120 and may be perpendicular to the first baffle crossbar 133, intersecting the first baffle crossbar 133 at about a center of the vessel 100 or cell culture surface 111 of the vessel 100. The second baffle crossbar 135 may comprise a vertical portion 131 on either end of the second baffle crossbar 135, with a retention tab 139 disposed at a topmost portion of each vertical portion 131. The vertical portion 131 may rise substantially vertically along the side walls 107 of the vessel 100, and the retention tabs 139 may releasably engage with recesses or baffle retention areas 119 at a top 117 of the side walls 107 that are sized and configured to retain the retention tabs 139 and secure the baffle 130 in the vessel 100. The second baffle crossbar may have a thickness T_{SBC}. The T_{SBC} may be about 0.05 in. The second baffle crossbar may have a length L_{SBC}. In some embodiments, the L_{SBC} may be about the same as or slightly shorter than W_{CCS}. In some embodiments, the L_{SBC} may be about 2.9 in. The distance D_{BRT} may denote the distance between retention tabs on opposite ends of the second baffle crossbar. In some embodiments, the D_{BRT} may be about 2.48 in.

The baffle 130 further comprises a third baffle crossbar 137 disposed at the port end 132 of the first baffle crossbar 133, the third baffle crossbar 137 arranged perpendicular to the first baffle crossbar 133. The third baffle crossbar 137 may be arranged horizontal to fluid flow from the port 120 and disposed at a front or port end 103 of the vessel 100. The third baffle crossbar 137 may act as a flow diverter 138, slowing the entrance of fluid from the port 120 across the cell culture surface 111. In some embodiments, the third baffle crossbar 137 comprises rounded shoulders 141 at either end of the third baffle crossbar 137 on a top portion of the third baffle crossbar 137. In some embodiments, the third baffle crossbar may extend over only a portion of the width of the cell culture area and may not extend fully to either sidewall. In such an embodiment, the third baffle crossbar may divert flow around a central area of the vessel.

In some embodiments, the third baffle crossbar may comprise feet on a bottom portion of the third baffle crossbar, the feet configured to contact the cell culture surface. In some embodiments, the third baffle crossbar may comprise two feet, one foot on either end of the crossbar. In some embodiments, the third baffle crossbar 137 comprises feet 145 at either end of the third baffle crossbar 137 on a bottom portion of the third baffle crossbar 137, wherein the feet 145 raise the third baffle crossbar away from the cell culture surface 111, and wherein the feet 145 are the only points of contact between the baffle and the cell culture surface 111. The feet may have a H_{F} and width W_{F}. In some embodiments, the Height is about 0.04 to about 0.05 in. In some embodiments, the W_{F} is about 0.07 in. The third baffle crossbar may have a width W_{TBC}. In some embodiments, the W-mc is less than W_{CCS}. In some embodiments, the W_{TBC} is about 2.4 in. The third baffle crossbar may have a height H_{TBC} from the bottom of the feet to the top portion of the third baffle crossbar. In some embodiments, the H_{TBC} is about 0.27 in. The third baffle crossbar may have a thickness T_{TBC}. In some embodiments, the T_{TBC} is about 0.06 in.

As described herein, the retention tabs of the baffle are sized and configured to be disposed within the baffle retention areas of side walls of a cell culture vessel. The baffle retention tabs may have a width W_{BRT}. In some embodiments, the W_{BRT} may be about 3.14 in. The baffle retention tab may have a width W_{RT} In some embodiments, the W_{RT} may be about 0.25 in. The baffle retention tab may have a thickness T_{RT} that allows the baffle retention tab to be retained within a recess or baffle retention area in a sidewall. In some embodiments, the T_{RT} may be about 0.09 to about 0.1 in. The retention tab may have a lower portion having height H_{LRT} from the bottom of the baffle. In some embodiments, the H_{LRT} is about 1.11 in. The retention tab may have an upper portion having a height H_{URT} from the bottom of the baffle. In some embodiments, the upper portion of the retention tab having H_{URT} is the topmost portion of the baffle, and thus, H_{URT} may correspond to the height of the baffle. In some embodiments, the H_{URT} is about 1.36 in.

Each baffle retention area may be a gap, recess, or cutout at a top portion of a side wall configured to receive and retain a retention tab of the baffle. In some embodiments, the baffle may comprise a retention tab at a top of a vertical portion of a baffle crossbar. In some embodiments, the baffle may comprise two retention tabs and the cell culture vessel may comprise two baffle retention areas. In some embodiments, the baffle may comprise three retention tabs and the cell culture vessel may comprise three baffle retention areas.

In some embodiments, the feet on the third baffle crossbar in conjunction with the retention tabs and height or length of the vertical portions of the baffle crossbars are configured to maintain a gap under the baffle to allow for movement of cells, medium, and spheroids into or out of the cell culture vessel. In some embodiments, the gap is about 0.040-0.050 inches.

In embodiments described herein, the crossbars forming the baffle are arranged in directions both perpendicular and parallel to fluid flow from the port. The crossbars may be elevated to permit liquid, cells, and/or spheroids to flow underneath the baffle unimpeded. For example, the feet and retention tabs of the baffle may be configured to allow for a gap D_{GB} from the bottom of the baffle to the bottom wall or cell culture surface of the cell culture vessel. In some embodiments, the D_{GB} is about 0.040-0.05 in. The width and length of the baffle may be configured to allow for a gap D_{GS} between the vertical portions of baffle crossbars and the non-port side walls of the cell culture vessel. In some embodiments, D_{GS} the is about 0.01 in.

In embodiments described herein, the design of the baffle and location of the baffle crossbars serve to visually segment the cell culture surface or growth surface into quadrants, assisting users when locating positions during microscopic examination, and permitting sampling from designated areas or quadrants. In some embodiments, a cap or lid placed on a top of the vessel side walls may serve to help retain the baffle in place.

Baffles, as described in embodiments herein, serve to stabilize movement of liquid culture medium within cell culture chamber. In particular, any movement of cell culture vessel with liquid culture medium stored therein may normally cause liquid culture medium to move within cell culture chamber due to the presence of open volume areas therein. However, with the baffle positioned within the cell culture vessel and sized to divide the cell culture surface into smaller quadrants instead of one large open area, the baffle is configured to inhibit the fluidity and/or free motion of liquid culture medium within cell culture chamber. Further stabilizing the baffle within cell culture chamber is the engagement between baffle retention tabs and baffle retention areas on the cell culture vessel sidewalls. Accordingly, baffles as described in embodiments herein are configured to preserve the condition of the cells received within the microcavities of the cell culture surface by inhibiting movement of liquid culture medium and isolating liquid culture medium from open volume areas within cell culture chamber.

In aspect of the subject matter described herein, a cell culture system is provided. The cell culture system may comprise a cell culture vessel, a baffle configured to be disposed within the cell culture vessel, and a protective carrier or support to protect the microcavities on a bottom wall of the cell culture vessel. In embodiments, the cell culture vessel may comprise a microcavity vessel. In some embodiments, the microcavity vessel is a microcavity flask, microcavity plate, microcavity bioreactor, or a stacked 3D microcavity culture vessel. In some embodiments, the microcavity vessel may be a microcavity flask. In some embodiments, the microcavity vessel may be a microcavity plate, such as an open well microcavity plate.

FIGS. 11-15 show an embodiment of a baffle 230 for a cell culture vessel 200 comprising an open well microcavity plate. Similar reference numbers are used to denote similar features described in other embodiments and figures. Instead of having top wall 115, such as shown in the vessel 100, such as the microcavity flask of FIGS. 1-5, the cell culture vessel 200 in FIGS. 11-15 is an open well plate.

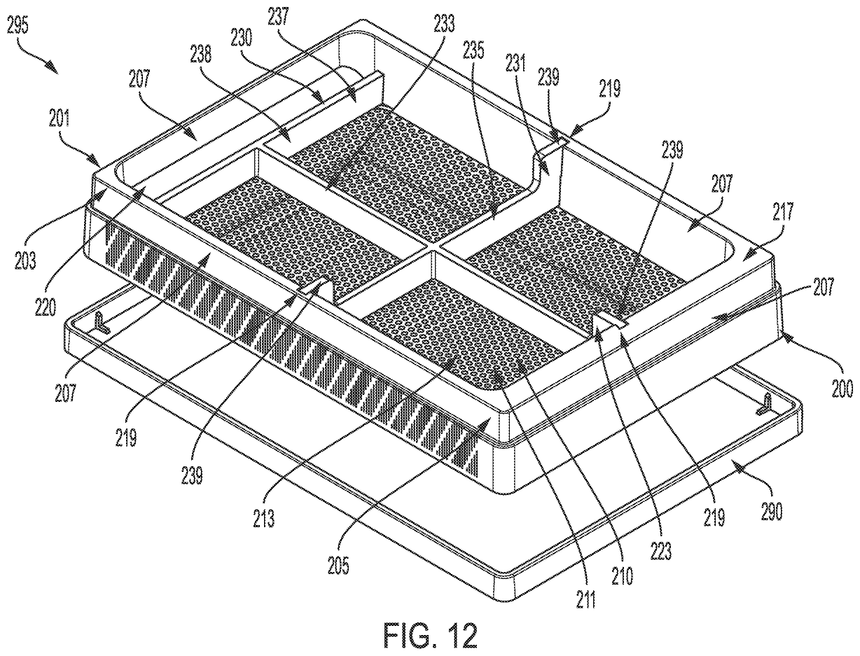

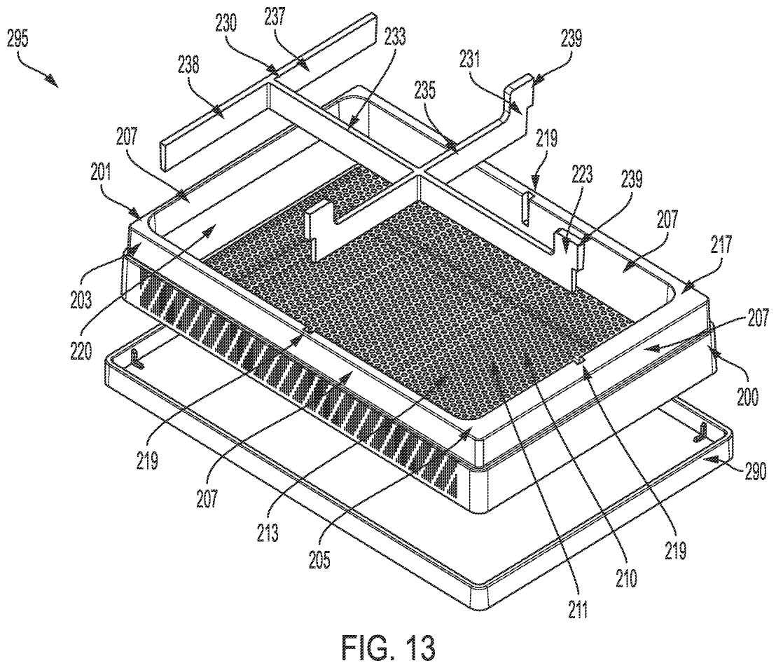

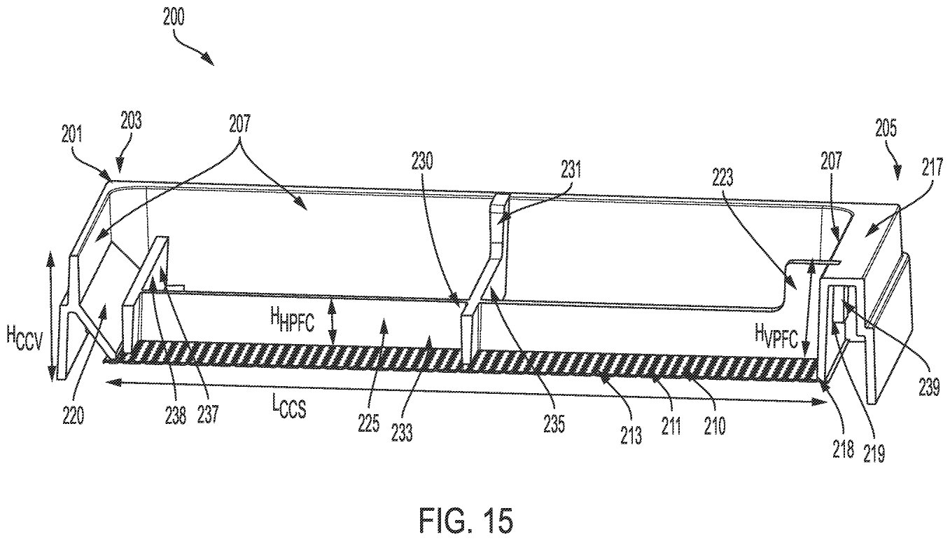

FIG. 11 shows an assembled cell culture system 295 comprising a microcavity vessel 200, a removable lid 250 disposed on top of the microcavity vessel 200, a baffle 230 disposed within the body 201 of the microcavity vessel 200, wherein the microcavity vessel 200 is disposed on a protective carrier 290. FIG. 12 shows partially disassembled components of the cell culture system 295 including the microcavity vessel 200 with baffle 230 disposed therein, wherein lid 250 has been removed, and wherein the microcavity vessel 200 is not disposed in the protective carrier 290. FIG. 13 shows disassembled components of the cell culture system 295 including the microcavity vessel 200 without lid 250, wherein the baffle 230 is not disposed within the microcavity vessel 200, and wherein the microcavity vessel 200 is not disposed within the protective carrier 290. FIG. 14 shows an embodiment of disassembled components of the cell culture system 295 including the microcavity vessel 200 without lid 250, wherein the baffle 230 is not disposed within the microcavity vessel 200, the baffle further comprising a handle 236 or pick-up point, wherein the microcavity vessel 200 is not disposed within the protective carrier 290. FIG. 15 shows a cross-sectional side view taken along a length of a cell culture vessel 200 with a baffle 230 disposed therein.

The cell culture vessel 200 may be a microcavity vessel, such as an open well plate, comprising a body 201 having a plurality of side walls 207 extending upwards from a bottom wall 210. The cell culture vessel 200 may have a height H_{CCV} defined by the height of the side walls 207. When lid 250 is placed on a top of the side walls 207, a cell culture chamber 204 is enclosed by the lid 250, side walls 207, and bottom wall 210. One of the side walls 207 may further comprise a port 220, such as a slanted surface along side wall 207, slanting downwards towards cell culture surface 211. The port 220 defines a port end 203 of the microcavity vessel 200 proximal to the port 220 and an opposite end 205 of the microcavity vessel 200 distal to the port 220. The microcavity vessel 200 includes a bottom wall 210 comprising a plurality of microcavities 213. The plurality of microcavities 213 may be referred to a microcavity substrate or cell culture surface 211 and may be gas permeable.

In some embodiments, the protective carrier 290 is a rigid plate that may protect the gas permeable microcavities 113 of microcavity vessels 200 during shipping and that may also act as a protective carrier for microcavity vessels during use for cell culture. The protective carrier 190 may be comprised of a rigid plate that is slightly larger than a footprint of a bottom of a microcavity vessel 200. In some embodiments, the protective carrier may be the protective carrier described in U.S. Patent Application No. 63/216,754, the contents of which are incorporated herein in their entirety.

The bottom wall 210 may be disposed at a bottom 218 of the side walls 207 and may define a cell culture surface 211 having a width W_{CCS} and length L_{CCS}. The cell culture surface 211 may comprise a plurality of microcavities 213. The cell culture vessel 200 may comprise a port 220 at one side wall 207 of the body 201, thereby defining a port end 203 of the vessel 200 proximal to the port 220 and an opposite end 205 distal to the port 220. The port 120 may be configured to provide fluid access into cell culture chamber 204 where a cell culture surface 211 is positioned.

The baffle 230 may comprise a first baffle crossbar 233, a second baffle crossbar 235, and a third baffle crossbar 237. The third baffle crossbar 237 may be disposed perpendicular to the first baffle crossbar 233 at an end of the first baffle crossbar 233 proximal to the port 220. The third baffle crossbar 237 may act as a flow diverter 238. In some embodiments, the third baffle crossbar 237 does not extend across a full width of the cell culture surface 211, but extends across only a portion of the width of the cell culture surface 211, thereby directing fluid flow from the port 220 away from the center area of the cell culture surface 211 to instead flow around either end of the third baffle crossbar 237.

The first baffle crossbar 233 may extend away from the third baffle crossbar 237, parallel to the cell culture surface 211 along a length L_{CCS} of the cell culture surface 211. The first baffle crossbar 233 may include a horizontal portion 225 having a height H_{HPFC} extending parallel to the cell culture surface 211 across a length of the cell culture surface 211 and a vertical portion 223 extending substantially vertically upwards from the horizontal portion 225 to a top portion 217 of the side wall 201. The vertical portion 223 has a height H_{VPFC} and extends along the side wall 207 at the opposite end 205 from the port end 203 of the vessel 200. The vertical portion 223 includes a retention tab 239 at a top of the vertical portion 233. As shown by the cross-section view of FIG. 15, the retention tab 239 extends or juts out horizontally from the vertical portion 223 towards the sidewall 207 and fits within, or is retained within, baffle retention area 219 formed by a gap or cutout in the side wall 207.

The second baffle crossbar 235 may intersect the first baffle crossbar 233 at about a center of the first baffle crossbar 233, wherein the second baffle crossbar 235 is perpendicular to the first baffle crossbar 233. The second baffle crossbar 235 may include a horizontal portion extending parallel to the cell culture surface 211 across a width of the cell culture surface 211 and a vertical portion 231 extending substantially vertically upwards from the horizontal portion, extending along the side walls 207. The vertical portion 231 includes a retention tab 239 at a top of the vertical portion 231, the retention tab 239 extending or jutting out horizontally towards the sidewall 207 and configured to be retained within a baffle retention area 219 formed by a gap or cutout in the side wall 207.

Gas permeability is a property that contributes to the 3D cell culture environment. By allowing for gas permeability within microcavities of the cell culture vessel, such as a microcavity vessel, cell culture growth media may need to be changed out less frequently and cell growth may be encouraged. Microcavity vessels are unique in their geometry and formation in that they are formed from a gas permeable substrate with micron-scale wells, also referred to as a microcavity substrate. Such microcavity vessels enjoy gas permeability due to the thickness of the microcavity substrate, wherein gas permeability occurs because the microcavity substrate is formed from a very thin polystyrene material of manufacture, which has a thickness of about 28 micrometers to about 72 micrometers. Though gas permeability may be an asset for culturing cell aggregates, the thinness of the microcavity substrate material makes microcavity cell culture vessels susceptible to damage during shipping and use.

In some embodiments, the protective carrier 290 is a rigid plate that may protect the gas permeable microcavities 113 of microcavity vessels 200 during shipping and that may also act as a protective carrier for microcavity vessels during use for cell culture. The protective carrier 190 may be comprised of a rigid plate that is slightly larger than a footprint of a bottom of a microcavity vessel 200. In some embodiments, the protective carrier may be the protective carrier described in U.S. Patent Application No. 63/216,754, the contents of which are incorporated herein in their entirety.

Each microcavity may include an inner cavity with a rounded bottom that is non-adherent to cells. Thus, microcavity vessels as described herein are cell culture devices facilitate 3D cell culture by allowing cells seeded into the microcavities to self-assemble or attach to one another to form a spheroid in each microcavity. Microcavities may be shallow and permit cell culture medium to cover the spheroids, organoids, or 3D cell aggregates in all cavities at once to make manual handling easy.

In an embodiment, a top plane of the microcavities may be recessed to a location close to a bottom of the side walls. Individual microcavities may hold a small volume of medium. The individual microcavities may have any suitable dimensions. For example, the diameter or width of individual microcavities may be in a range of about 500 microns to about 5 millimeters. The depth of individual microcavities may be in a range of about 500 microns to about 6 millimeters. In some embodiments, a depth of the individual microcavities may be about 500 microns to about 650 microns. In some embodiments, a depth of the individual microcavities may be about 1.6 millimeters. An excess of culture medium may be added to the microcavity vessel so that the spheroids, organoids, or 3D cell aggregates do not need to rely only on the small amount of medium in the individual microcavities.

In some embodiments, the microcavity substrate may have a cross-sectional shape that is undulating or is a shape approximating a sine wave. In such embodiments, the bottom of the microcavity well is rounded (e.g., hemispherically round), the side walls increase in diameter from the bottom of the well to the top and the boundary or barrier between wells is rounded. As such, the top of the microcavity wells does not terminate at a right angle. In some embodiments, the width of the well is greater than the width of the barrier between contiguous wells. Such an embodiment permits a greater number of wells within a given area of culture surface.

In some embodiments, the plurality of microcavities are arranged in a hexagonal close-pack pattern. In some embodiments, each microcavity comprises a rounded bottom. In some embodiments, each microcavity is configured such that cells cultured in the microcavity vessel form three-dimensional (3D) cell aggregates. In some embodiments, an interior surface of the microcavity substrate is non-adherent to cells. In some embodiments, the interior surface of the microcavity substrate comprises a cell non-adherent surface coating comprising perfluorinated polymers, olefins, lipids, agarose, non-ionic hydrogels, polyethers, polyols, polymers that inhibit cell attachment, or a combination thereof. In some embodiments, the cell non-adherent surface coating comprises an ultra-low attachment (ULA) surface coating.

The microcavity substrate, microcavity vessel, and baffle may be formed from the same material or a similar material. In some embodiments, the microcavity substrate may be molded or formed separately from the rest of the microcavity vessel and bonded subsequently through thermal-bonding, ultrasonic welding, or any other method of plastic joining. The material of construction for the microcavity vessel, microcavity substrate, and/or baffle may comprise a “plastic” polymer, co-polymer, or polymer blend. Nonlimiting examples include silicone rubber, polystyrene, polypropylene, polyethylene, polyethylene terephthalate, polymethylpentene, polycarbonate, polymethyl methacrylate, styrene-ethylene-butadiene-styrene, other such polymers, or a combination thereof. In some embodiments, the microcavity substrate is formed from polydimethylsiloxane (PDMS), polymethylpentene, (poly)4-methylpentene (PMP), polyethylene (PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof. Any suitable construction method may be used to form the microcavity substrate, microcavity vessel, and baffle, such as nonlimiting examples including injection molding, thermoforming, 3D printing, or any other method suitable for forming a plastic part.

In some embodiments, the protective carrier is formed from a polymer, metal, or glass. In some embodiments, the polymer comprises polystyrene, polypropylene, polyethylene, polyethylene terephthalate, polymethylpentene, polycarbonate, polymethyl methacrylate, styrene-ethylene-butadiene-styrene, other such polymers, or a combination thereof. In some embodiments, the metal comprises aluminum, stainless steel, zinc, or a combination thereof. In some embodiments, the glass comprises a borosilicate glass. In some embodiments, the protective carrier is formed from a recyclable material. In some embodiments, the protective carrier is formed from a biodegradable material. In some embodiments, the protective carrier is opaque. In some embodiments, the protective carrier is translucent.

The above-described baffles, cell culture vessels including baffles positioned within the respective cell culture chambers of microcavity cell culture vessels, and systems for microcavity cell culture including baffles positioned within the respective cell culture chambers of microcavity cell culture vessels minimize the amount of movement of liquid culture medium within a cell culture chamber. The baffles as described herein are capable of being inserted, removed, and/or repositioned within a cell culture vessel to facilitate both the receipt of a liquid medium along the cell culture surface of the vessel and the stabilization of the liquid medium relative to the cell culture surface. Accordingly, baffles as described herein are configured to preserve the condition of the cells received within the microcavities of the cell culture surface by creating smaller sections within the cell culture chamber and thereby inhibiting movement of liquid culture medium and preventing liquid culture medium from expanding to one large open volume area within cell culture chamber.

Based on the foregoing, it should be understood that the baffles as described herein may be used to stabilize a liquid medium contained within the cell culture vessel when physically maneuvering and/or transporting the vessel, thereby minimizing the amount of movement of the liquid medium within the vessel and reducing the potential for turbulence against the cells being cultured along the cell culture surface of the vessel.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

What is claimed is:

1. A cell culture vessel, comprising:
a vessel body comprising a bottom wall and a plurality of side walls that define a cell culture chamber, the bottom wall comprising a cell culture surface;
a baffle configured to be disposed parallel to the cell culture surface within the vessel body, the baffle comprising:
a first baffle crossbar extending across a length of the cell culture surface;
a second baffle crossbar intersecting the first baffle crossbar; and
a third crossbar disposed at an end of the first baffle crossbar.

2. The cell culture vessel of claim 1, wherein the second baffle crossbar is disposed perpendicular to the first baffle crossbar.

3. The cell culture vessel of claim 1, wherein the second baffle crossbar extends across a width of the cell culture surface.

4. The cell culture vessel of claim 1, wherein the third crossbar is disposed perpendicular to the first baffle crossbar.

5. The cell culture vessel of claim 1, wherein the third baffle crossbar extends across a portion of a width of the cell culture surface.

6. The cell culture vessel of claim 1, wherein the third baffle crossbar is a flow diverter.

7. The cell culture vessel of claim 1, wherein the third baffle crossbar comprises rounded shoulders on a top of the third baffle crossbar at either end of the third baffle crossbar.

8. The cell culture vessel of claim 1, wherein the third baffle crossbar comprises feet in contact with the cell culture surface to provide a gap where the horizontal portion of the third baffle crossbar is raised away from the cell culture surface.

9. (canceled)

10. The cell culture vessel of claim 1, wherein the cell culture surface comprises a plurality of microcavities.

11. The cell culture vessel of claim 1, wherein the cell culture vessel further comprises a port disposed at one side wall of the plurality of side walls, wherein the port is configured for aspiration and media exchange.

12. The cell culture vessel of claim 11, wherein the third baffle crossbar is arranged proximal to the port and perpendicular to fluid flow from the port.

13. The cell culture vessel of claim 11, wherein the cell culture vessel further comprises a cap or lid removable to provide access to the port.

14. (canceled)

15. The cell culture vessel of claim 1, wherein the vessel body further comprises atop wall and wherein the cell culture vessel further comprises a lid configured to releasably attach to a top portion of the sidewalls to enclose the cell culture chamber.

16. The cell culture vessel of claim 1, wherein a plurality of baffle retention areas are disposed at a top portion of side walls of the vessel.

17. The cell culture vessel of claim 16, wherein the baffle further comprises a plurality of retention tabs configured for insertion into corresponding baffle retention areas.

18. The cell culture vessel of claim 17, wherein the first baffle crossbar comprises a horizontal portion and a vertical portion, wherein a retention tab is disposed at a top of the vertical portion.

19. The cell culture vessel of claim 17, wherein the second baffle crossbar comprises a horizontal portion and vertical portions on either end of the horizontal portion, wherein a retention tab is disposed at a top of each vertical portion.

20-22. (canceled)

23. The cell culture vessel of claim 1, wherein a bottom of the baffle is raised about 0.04 in to about 0.5 from the cell culture surface.

24. The cell culture vessel of claim 1, wherein an end of the first baffle crossbar is angled to correspond to a draft angle of a corresponding side wall of the cell culture vessel.

25. (canceled)

26. The cell culture vessel of claim 1, wherein the vessel body is configured to receive a liquid culture medium within the cell culture chamber, wherein the baffle is configured to inhibit movement of the liquid culture medium across the cell culture surface.

27. The cell culture vessel of claim 1, wherein the baffle is removable.

28-30. (canceled)
